(19) United States
(12) Patent Application Publication
(10) Pub. No.: US 2002/0049160 A1
(43) Pub. Date: Apr. 25, 2002

(54) New strategy for drug design

(76) Inventors: Li-Yen Mae Huang, Galveston, TX (US); Yanping Gu, League City, TX (US)

Correspondence Address: MUETING, RAASCH & GEBHARDT, P.A., P.O. BOX 581415, MINNEAPOLIS, MN, 55458, US

(21) Appl. No.: 09/934,246

(22) Filed: Aug. 21, 2001

Related U.S. Application Data
(60) Provisional application No. 60/226,833, filed on Aug. 22, 2000.

(51) Int. Cl. A61K 38/17; C12Q 1/00

(52) U.S. Cl. 514/12; 435/4

(57) ABSTRACT

The present invention provides methods for identifying an agent that alters, preferably increases, an NMDA receptor mediated response in a neuron. The method includes contacting the neuron with the agent, where the neuron includes an NMDA receptor and an elevated level of PKC. The neuron can be ex vivo, in vitro, or in vivo. The present invention also provides methods for identifying an agent that, in an animal, reduces pain from a neuropathological condition.

### CONTINUING APPLICATION DATA

This application claims the benefit of U.S. Provisional Application Ser. No. 60/226,833, filed Aug. 22, 2000, which is incorporated by reference herein.

### GOVERNMENT FUNDING

The present invention was made with government support under Grant No. NS30045 and NS 11255, awarded by the National Institutes of Health. The Government has certain rights in this invention.

### BACKGROUND

The drug 1-(aminomethyl)cyclohexaneacetic acid, also known as Gabapentin (GBP), was originally designed as an antiepileptic drug for seizure patients. It unexpectedly provided significant relief of ongoing spontaneous and paroxysmal pain in patients with peripheral nerve injuries or central lesions. In addition, GBP is efficacious for treating postherpetic neuralgia, diabetic neuropathy and trigeminal neuralgia. Because a combined therapy of opiates and GBP can produce much more effective pain relief in patients, GBP is used frequently with opiates to treat severe cancer pain.

The mechanisms underlying the antinociceptive actions of GBP remain elusive (Taylor et al., Epilepsy Res., 29:233-249 (1998). Although GBP is a structural analog of γ-aminobutyric acid (GABA), it does not interact with either GABA_{A} or GABA_{B} receptors. GBP was found to interact with system L-amino acid transporter, thus altering the synthesis and release of GABA in the brain. However, blocking the transporter does not reverse the anti-allodynic effect of GBP. More recently, GBP was shown to bind to the α₂β subunit of voltage-activated Ca²⁺ channels with high affinity. Nevertheless, GBP can inhibit N and P/Q subtypes of Ca²⁺ channel activity only by a very moderate amount (<20%) and its effects on L-type channels are not consistent in different preparations.

Because N-Methyl-D-Aspartate (NMDA) receptors play a central role in the sensitization of nociceptive neurons, the actions of GBP on NMDA receptors have also been examined. The results again are varied. At the cellular level, GBP increases NMDA-evoked currents in some cultured cortical cells without added glycine, but does not affect NMDA responses with 1 μM glycine. In spinal cord slices, GBP increases the amplitude of NMDA receptor-mediated excitatory postsynaptic currents (EPSCs) in deep, but not in superficial dorsal horn cells. Behavioral studies show that GBP does not affect transient responses to noxious heat or mechanical stimuli but potently blocks sustained nociceptive responses elicited by inflammatory agents, e.g., formalin. Furthermore, administration of serine, an agonist for the glycine site at NMDA receptors, blocks the antinociceptive effects of GBP.

While GBP is useful for the treatment of pain, GBP can cause dizziness, somnolence, and other symptoms and signs of central nervous system depression. Moreover, GBP use was fetotoxic in rodents and was associated with postimplantation fetal loss in rabbits. Thus, there is a need for new agents that are useful in the treatment of pain.

### SUMMARY OF THE INVENTION

This invention represents a significant advance in the art of identifying agents that can be used to modify an NMDA receptor mediated response in certain neurons, especially those involved in the transmission of pain. Such agents would be more efficacious, specific, and have fewer side effects. It has been observed that it is possible to alter the NMDA receptor response in neurons that contain elevated levels of protein kinase C (PKC), but not alter the response in neurons that do not contain the elevated levels of PKC. It is believed that neurons containing NMDA receptor and elevated PKC levels are sensitized to painful stimuli, resulting in hyperalgesia, allodynia, and/or persistent pain. Thus, the present invention provides a method for identifying an agent that increases an NMDA receptor mediated response in a neuron to an agonist. The neuron includes an NMDA receptor and an elevated level of PKC. The method includes contacting the neuron with the agent and determining whether the NMDA receptor mediated response in the neuron contacted with the agent is altered relative to a neuron not contacted with the agent.

The neuron can be an ex vivo neuron. For instance, the neuron can be one that is isolated from a spinal cord, including, for instance, from lamina I, lamina II, lamina IV, or lamina V of a dorsal horn, or from lamina X of a central canal. The neuron can be isolated from a brain, including, for instance, from a trigeminal subnuclear caudalis. The neuron can be an in vitro neuron. For instance, the neuron can be a human neuroblastoma neuron, including NG108-15, N1E-115, or SHSY5Y. The agonist can be, for instance, NMDA, glutamate, or aspartate. The method can further include determining whether the agent increases the affinity of glycine for the NMDA receptor.

The present invention also provides a method for identifying an agent that, in an animal, reduces pain from a neuropathological condition. The method includes contacting a neuron with the agent and determining whether the NMDA receptor mediated response in the neuron contacted with the agent is altered relative to a neuron not contacted with the agent. The neuron typically includes an NMDA receptor and an elevated level of PKC, The neuropathological condition can include, for instance, peripheral nerve injury, postherpetic neuralgia, diabetic neuropathy, trigeminal neuralgia, or cancer. The neuron can be an ex vivo neuron or an in vivo neuron. The alteration in the NMDA receptor mediated response can be measured by evaluating a change in allodynia or in hyperalgesia in an animal

Definitions

As used herein, the term “NMDA receptor” refers to a class of glutamate receptor that is activated by N-methyl-D-aspartate (NMDA), as well as other molecules including glutamate and aspartate. “Activation” of an NMDA receptor refers to the opening of the channel upon interacting with, for instance, NMDA, glutamate, or aspartate. As used herein, an “NMDA receptor mediated response” refers to the opening of NMDA receptor channels when the NMDA receptor interacts with an agonist. This allows the rapid influx of cations including Na⁺, K⁺, and/or Ca⁺⁺ ions into the neuron, thus resulting in the depolarization of the cell.

As used herein, the term “agonist” refers to a compound that activates an NMDA receptor. An agonist can be a naturally occurring compound (e.g., a compound produced by an animal, including for instance glutamate or aspartate) or a synthetic compound (e.g., NMDA).

As used herein, the term “neuron” refers to a cell responding to sensory and/or electrical stimuli generating action potentials and conducting electrical activity to another cell. Typically, a neuron includes an NMDA receptor. The cell can be in vitro (i.e., a cultured cell line), ex vivo (i.e., a cell that has been removed from the body of an animal), or in vivo (i.e., within the body of an animal). A neuron can be an inhibitory or an excitatory neuron. An inhibitory neuron is one that releases inhibitory transmitters, including, for instance, γ-aminobutyric acid (GABA) or glycine. An excitatory neuron is one that releases excitatory transmitters, including, for instance, glutamate or aspartate.

As used herein, the term “injured neuron” and “injured cell” refers to a neuron that been exposed to a trauma, including, for instance, viral infection, a direct blow, transection of nerve fibers, or exposure to chemicals released from surrounding cells as a result of inflammation, including, for instance, prostaglandins, bradykinin, histamine, or capsaicin. Typically, an injured neuron is sensitized to painful stimuli.

As used herein, the term “sensitized neuron” or “sensitized cell” refers to a neuron that has been altered such that activation of the neuron results in a response that is greatly enhanced relative to a non-sensitized neuron. Sensitized neurons play a role in allodynia or hyperalgesia. As used herein, the term “allodynia” refers to an increased sensitivity to a stimulus that was previously innocuous. For instance a stimulus that was previously innocuous is now considered noxious, i.e., painful. As used herein, the term “hyperalgesia” refers to an increased sensitivity to a noxious stimulus. Allodynia and hyperalgesia can be primary or secondary. Primary allodynia and primary hyperalgesia mean the location of the increased sensitivity is at the same site as an injury. Secondary allodynia and secondary hyperalgesia mean the location of the increased sensitivity is at a site that is not identical to the site of the injury. A sensitized neuron typically has higher amounts of protein kinase C and nitric oxide synthase than neurons that are not sensitized.

The term “elevated level of PKC” is described in detail herein.

As used herein, the term “neuropathological condition” refers to functional disturbances and/or pathologic changes in an animal's nervous system. Examples of functional disturbances include persistent pain in an inflammatory state, arthritis, peripheral nerve injury, brain injury, spinal cord injury, cancer, neuralgia (including, for instance, postherpetic neuralgia and trigeminal neuralgia) and neuropathy (including, for instance, diabetic neuropathy). Examples of pathological changes include the presence of persistent pain due to functional disturbances. As used herein, the term “persistent pain” can refer to pain that is constant, for instance the type of pain associated with cancer or back pain. Alternatively, persistent pain can also refer to pain that continues for at least about 10 minutes after an initial mechanical stimulus causing the pain is removed.

Unless otherwise specified, the indefinite article “a” or “an” means one or more.

### BRIEF DESCRIPTION OF THE FIGURES

FIG. 1. Effect of GBP on NMDA-evoked currents in control cells. A. NMDA-evoked currents measured at −60 mV in control, 100 μM GBP and wash. B. Bar graphs summarize data from 6 cells. Relative NMDA-evoked currents (I/Iₒ), expressed in percentages, were normalized to the currents measured in control conditions. I_peak, peak NMDA response; I_ss, steady state NMDA response; ms, millisecond; pA, picoamps; I/Iₒ, current measured in test conditions (I) relative to current in the control (Iₒ).

FIG. 2. Effect of GBP on NMDA responses in cells with added PKC. A. Sample traces of NMDA-evoked currents recorded with PKCM (0.025 U/ml) in the patch pipette. The label above each trace indicates the condition and time when the record was taken. B. Time course of NMDA responses in the presence of PKCM and GBP. Data points are average NMDA responses from 5 cells. Error bars indicate SEM. The periods of PKCM and GBP applications are indicated by horizontal bars. C. Bar graphs showing the potentiation of NMDA currents by GBP. I_peak, peak NMDA response; I_ss, steady state NMDA response; ms, millisecond; pA, picoamps; I/Iₒ, current measured in test conditions (I) relative to current in the control (Iₒ).

FIG. 3. The effect of the PKC inhibitor, chelerythrine, on GBP potentiation of NMDA responses in cells with added PKC. A. Time course of NMDA responses when chelerythrine was added to the external solution and PKCM to the pipette solution. The letters, i.e., 1, 2 and 3, in the time course plot correspond to traces shown above in A. The periods of PKCM, Chelery, and GBP applications are indicated by horizontal bars. Filled circles, peak currents; empty circles, currents measured at to end of a 2 second application of NMDA. B. Summary of data from 10 cells. I_peak, peak NMDA response; I_ss, steady state NMDA response; ms, millisecond; pA, picoamps; I/Iₒ, current measured in test conditions (I) relative to current in the control (Iₒ), I(pA), current in picoamps.

FIG. 4. Effect of GBP on NMDA responses in inflamed rats without PKC treatment. A. Representative NMDA current traces from a dorsal horn cell isolated from a CFA-treated rat. B. Bar graphs showing the potentiation of NMDA currents by GBP in inflamed rats without PKC treatment. The peak and steady stated currents were enhanced by 28.0±2.0% and by 27.0±3.3% respectively (n=19)(P<0.05). I_peak, peak NMDA response; I_ss, steady state NMDA response; ms, millisecond; pA, picoamps; I/Iₒ, current measured in test conditions (I) relative to current in the control (Iₒ).

FIG. 5. Effect of GBP on glycine affinity for NMDA receptors. A. The potentiation of NMDA responses by GBP depends on external glycine concentration. In the example shown, GBP increased peak NMDA currents by 40% with 0.2 μM external glycine, but had no effect on NMDA responses with 2 μM glycine. B. Dose-response curves for glycine with and without GBP. Under control conditions, the EC₅₀ of glycine for NMDA receptors was 0.2 μM. In the presence of 50 μM of GBP, the EC₅₀ was reduced to 0.09 μM (n=4).

### DETAILED DESCRIPTION OF THE INVENTION

The present invention is directed to a method for identifying an agent that alters an NMDA receptor mediated response in a neuron. Another aspect of the invention is directed to a method for identifying an agent that reduces in an animal pain from a neuropathological condition. The neuron used in the method can be cat, dog, rat, mouse, or human, preferably human. The neuron can be in vitro, ex vivo, or in vivo, preferably in vitro or ex vivo. Examples of useful neurons that can be used in vitro include cultured neuroblastoma cells, preferably rat, mouse, or human, more preferably human. An example of a cultured neuroblastoma cell is NG108-15 (available from ATCC under ATCC number HB-12317), N1E-115 (available from ATCC under ATCC number CRL 2263), and SHSY5Y (available from ATCC under ATCC number ATCC CRL 2266). Examples of useful ex vivo neurons include neurons that are involved in the sensation of pain. Preferably, an ex vivo neuron is removed from a rat or a mouse. For instance, useful neurons can be removed from the spinal cord of an animal, including, for instance, the dorsal horn or cells surrounding the central canal. Preferably, such neurons are removed from lamina I, lamina II, lamina IV, or lamina V of the dorsal horn. Other useful ex vivo neurons include neurons from the brain, including, for instance, neurons in trigeminal subnucleus caudalis or in the periaqueduct grey. Preferably, such neurons are removed from dorsal horn of the subnucleus. Examples of useful in vivo neurons include neurons that are involved in the transmission of pain or nociceptive stimuli.

The method includes contacting a neuron with the agent, the neuron including an NMDA receptor, and determining whether the NMDA receptor mediated response in the neuron contacted with the agent is altered relative to a neuron that is not contacted with the agent. Neurons in vitro and ex vivo can be contacted directly with the agent by, for instance, adding the compound to the media in which the neuron is incubated. Neurons in vivo can be contacted directly with an agent by administering the agent to the animal.

The invention is not intended to be limited by the types of agents that can be screened for activity using the methods described herein. Accordingly, an agent can be, for instance, an amino acid, a polypeptide, an organic molecule, polyketide, or non-ribosomal peptide. Agents useful in the methods of the present invention can be produced by natural organisms, or produced using methods known to the art, including recombinant techniques, or chemical or enzymatic synthesis techniques.

Preferably, a neuron used in the methods of the present invention has an elevated level of PKC relative to a neuron having a normal level of PKC. An elevated level of PKC is a concentration of PKC that is preferably at least about 1.5-fold, more preferably at least about 2-fold, most preferably at least about 2.5-fold higher than the PKC concentration in a normal neuron.

In some aspects of the invention, the level of PKC can be elevated by adding PKC to the neuron as described in the Examples. Alternatively, neurons that have elevated levels of PKC can be used. This can be accomplished by removal of neurons from an animal that has undergone exposure to a stimulus that causes the sensation of pain, for instance from inflammation, in non-neural tissue. For instance, the Examples describe the subcutaneous administration of formalin to a footpad to cause inflammation and nociception. Other stimuli that can be used include carrageenan, or a kaolin/carrageenan mixture. Preferably, the animal displays allodynia or hyperalgesia as a result of the stimulus. Alternatively, neurons can be injured prior to removal from an animal. Injury includes, for instance, viral infection by a virus that targets neurons, or ligation or cutting of spinal nerves. Alternatively, a neuron can be manipulated after removal so that it is injured. For instance, a neuron can be stretched or treated with inflammatory agents including, for instance, prostaglandins, bradykinin, histamine, or capsaicin. Preferably, when in vivo neurons are used, the animal has been manipulated to elevate the levels of PKC in neurons involved in the sensation of pain. For instance, the animal can be subcutaneously administered formalin, or spinal nerves can be cut or ligated. Alternatively, instead of using a neuron having elevated levels of PKC, in some aspects of the invention it is expected that a chemical, for instance a phorbol ester, can be added to the neuron to cause the phosphorylation of polypeptides in the neuron, preferably the NMDA receptor.

Typically, NMDA receptors present in the neuron are activated by exposing the neuron to an NMDA receptor agonist, preferably NMDA, glutamate, or aspartate, more preferably NMDA. Activation of the neuron can occur before, at the same time, or after the neuron is exposed to the agent. In those aspects of the invention using in vitro or ex vivo neurons, the agonist is added to the media in which the neuron is incubated. Conditions necessary for activation of an NMDA receptor present in an in vitro or ex vivo neuron are known to the art. When the neuron is in vitro or ex vivo, an NMDA receptor is activated by the addition of, in increasing order of preference, at least about 10 μM NMDA, at least about 20 μM NMDA, at least about 50 μM NMDA, or most preferably, at least about 100 μM NMDA. Preferably, no greater than about 500 μM NMDA is added.

An NMDA receptor mediated response can be measured by methods known to the art. Preferably, an NMDA receptor mediated response in an in vitro or ex vivo neuron is measured by whole-cell patch clamp (see, for instance, Hamill et al., Pflugers Arch., 391:85-100 (1981) or Rae et al., J. Neurosci, Methods, 37:15-26 (1991)). Other methods that can be used include sharp microelectrode recording, and methods using calcium sensitive dyes such as Fura-2. Typically, a neuron is considered to have had an NMDA receptor mediated response when there is a difference in current amplitude and/or kinetics (for instance shape of currents) before and after NMDA application.

In in vitro or ex vivo neurons, the level of the NMDA receptor mediated response in a neuron exposed to an agent is determined and compared to the level of an NMDA receptor mediated response in a neuron that was not exposed to the agent. An agent that causes a change in the level of the NMDA receptor mediated response that is, in increasing order of preference, at least about 5%, at least about 10%, at least about 15%, most preferably at least about 20% higher or lower than a neuron not exposed to the agent is considered to be an agent that alters an NMDA receptor mediated response in a neuron. Preferably, an agent increases the NMDA receptor mediated response when the neuron is an inhibitory neuron. Preferably, an agent decreases the NMDA receptor mediated response when the neuron is an excitatory neuron. Whether a neuron is inhibitory or excitatory can be evaluated using methods known to the art, including, for instance, immunocytochemistry.

Activation of an NMDA receptor present in an in vivo neuron is typically accomplished by exposing the animal to an innocuous stimulus (to measure, for instance, allodynia) or a noxious stimulus (to measure, for instance, hyperalgesia). An NMDA receptor mediated response in an in vivo neuron is typically measured by evaluating the response of an animal to an innocuous or a noxious stimulus. Methods for evaluating the response are known to the art. The animal used is one that has been manipulated to elevate the levels of PKC in neurons involved in the sensation of pain. If the animal's response to an innocuous or a noxious stimulus is increased, the animal is considered to be displaying allodynia or hyperalgesia. It can be inferred from the presence of allodynia or hyperalgesia that the stimulus caused an NMDA receptor mediated response in a neuron.

In in vivo neurons, the responsiveness of the animal to an innocuous or noxious stimulus is compared to the responsiveness of an animal that did not receive the agent. An agent that causes a statistically significant change in the responsiveness and can be blocked by an NMDA receptor antagonist (for instance MK801 (5-methyl-10,11-dihydro-5H-dibenzo[a, d]cyclohepten-5,10-imine maleate), APV (2-amino-5-phosphonovaleric acid), or AP5 (2-amino-5-phosphonopentanoic acid)) is considered to be an agent that alters an NMDA receptor mediated response in a neuron. Preferably, an agent decreases the responsiveness of an animal to an innocuous or noxious stimulus. An agent that decreases the responsiveness of an animal to an innocuous or noxious stimulus is expected to be useful for reducing, in an animal, pain from a neuropathological condition.

In other aspects, the methods of the present invention can be used to evaluate whether an agent alters the affinity of glycine for an NMDA receptor. Typically, increased glycine affinity for an NMDA receptor results in an increased NMDA receptor mediated response, and decreased glycine affinity for an NMDA receptor results in a decreased NMDA receptor mediated response. Preferably, an agent increases the affinity of glycine for an NMDA receptor in an inhibitory neuron, or decreases the affinity of glycine for an NMDA receptor in an excitatory neuron. Preferably, the effect of glycine is altered by the presence of glycine receptor blockers, including, for instance, 7-Cl (7-chlorokynurenic acid).

The methods of the present invention can also be used to identify agents that act selectively on neurons that have an elevated level of PKC. For instance, the methods can be used to identify an agent that increases an NMDA receptor mediated response by contacting a neuron having an elevated level of PKC with the agent. The NMDA receptor mediated response in the neuron can be determined and compared to a neuron that was contacted with the agent but does not contain an elevated level of PKC.

It is expected that agents that are able to alter, preferably increase, an NMDA receptor mediated response in a neuron having an elevated level of PKC can be used in methods of therapeutic and/or prophylactic treatment of animals having a neuropathological condition. For instance, an agent identified in the methods of the present invention can be used to reduce nociception, allodynia, or hyperalgesia in an animal. Such methods typically include administering to an animal a therapeutic amount of an agent such that a symptom of the neuropathological condition is decreased or prevented. The methods can further include administration of a second compound, preferably an opiate.

The present invention is illustrated by the following examples. It is to be understood that the particular examples, materials, amounts, and procedures are to be interpreted broadly in accordance with the scope and spirit of the invention as set forth herein.

### EXAMPLE 1

Cell isolation:

Single dorsal horn neurons were isolated from the lamina I and II of the lumbar spinal cord of 13-20 days old Long-Evans rats. After the rat had been anaesthetized with pentobarbitone (0.1 mg/g), the lumbar region of the spinal cord was rapidly removed from the animal and put into an ice-cold, oxygenated dissecting solution. The solution consisted of (in mM): NaCl (120), KCl (10), CaCl₂ (1), MgCl₂ (6), glucose (10), and HEPES (10) (pH 7.15). The osmolarity was 305-315 mosm. The tissue was cut into 300 μm transverse or horizontal slices with a vibratome slicer and incubated in the dissecting solution at 34.5° C. for 30 minutes. The slices were then transferred to a new dissecting solution that contained 2.7 units/ml papain (Sigma P-3250). After 40-60 minutes incubation at 34.5° C., the tissue was washed with enzyme-free dissecting solution and stored at room temperature. Prior to an experiment, neurons from a slice were dissociated by triturating the tissue with a series of fire-polished Pasteur pipettes.

Induction of inflammation: Some experiments were performed on dorsal horn neurons isolated from inflamed rats (15-25 days old, Sprague-Dawley rats). To induce inflammation, 0.1 ml complete Freund Adjuvant (CFA, Mycobacterium tuberculosis, suspended in an oil/saline 1:1 emulsion; 1 mg Mycobacterium/ml) was injected subcutaneously into the plantar surface of one rat hindpaw. The paw started to swell 24 hours later, and the swelling persisted for about two weeks. Two to seven days after the CFA injection, the superficial layer of the lumbar (L4-L6) cord ipsilateral to the injection site was dissected out and neurons were isolated using the same method as described above.

Electrical recordings: NMDA-receptor mediated currents in normal dorsal horn neurons were examined using the whole-cell patch clamp recording technique at room temperature (20-23° C.). The pipette solution contained (in mM): 120 cesium methanesulphonate, 20 CsCl, 1 CaCl₂, 5 BAPTA, 1 MgCl₂, 10 HEPES, 2.5 Na₂ATP, adjusted to pH 7.2 with CsOH. The external solution contained (mM): NaCl,140; KCl, 4; CaCI₂, 2.5; MgCl₂, 0.02; HEPES,10; Glucose,10; Sucrose,10; adjusted to pH 7.4 with KOH. For inflamed neurons, perforated patch electrodes were used to record NMDA responses. The electrode was first dipped into a solution containing (in mM) 100 KMeSO₃, 20 KCl, 10 HEPES, 1 BAPTA, 0.5 CaCl₂, 10 glucose, adjusted to pH 7.2 with KOH, and then back filled with the same solution containing amphotericin B (240 μg/ml). NMDA was delivered to the recorded cell using the fast perfusion technique (Gu et al., Neuron, 6:777-784 (1991)) or with pressure-feed glass pipettes. PKC was included in the pipette solution in some experiments. The resistances of patch pipettes were <20 Mohms. Current recordings were made with an Axopatch-200A patch clamp amplifier (Axon Instruments, Foster City, Calif.). The recorded whole-cell currents were filtered at 2 kHz and sampled at 200 μs per point. The average data are expressed as mean±SEM. Statistical significance of the data was evaluated using Student's t-test or one-way ANOVA. A level of P<0.05 was considered statistically significant. Dose-response curves were fitted according to the Hill equation, i.e., I/Iₘₐₓ=[Gly]ⁿ/[Gly]ⁿ+[EC₅₀]ⁿ where I is the measured current, Iₘₐₓ is the current measured at the saturated dose (2 μM) of glycine, n is the Hill coefficient, EC₅₀ is the concentration of glycine used when the response is 50% of maximal.

All chemicals were of ultrapure grade. GBP used for this study was a gift from Parke-Davis Pharmaceutical Research (Ann Arbor,Mich.). The concentrations of chemicals used were as follows: 100 μM NMDA, 0.1-6 μM glycine, 50 or 100 μM GBP, 0.0125 U/ml or 0.025 U/ml PKCM, 6.6 μM chelerythrine.

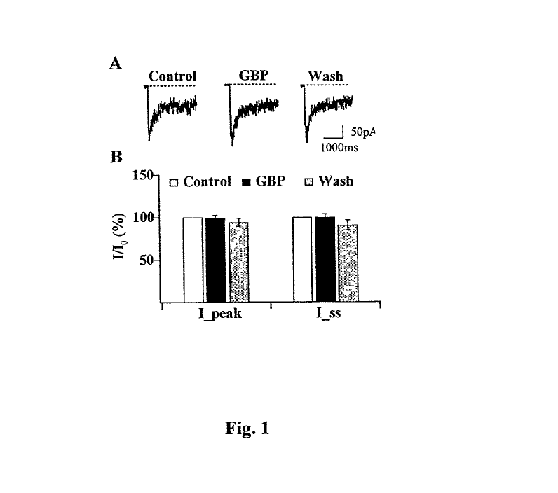

GBP had no effect on NMDA-activated currents under normal conditions. The effect of GBP on NMDA receptor currents in neurons isolated from control rats was examined (FIG. 1). In the presence of 0.2 μM external glycine, a 2 second-application of NMDA (100 μM) to a dorsal horn neuron produced an inward current that peaked rapidly and then decayed to a steady state (FIG. 1A). This current could be blocked by the NMDA-receptor antagonist, APV (20 μM), and the I-V curve of the currents had a characteristic negative-resistance region, a result of voltage-dependent block of external Mg²⁺ (Chen et al., Nature 356:521-523 (1992)). Extracellular application of GBP (100 μM) changed neither the peak (I/I₀=0.99±0.04, n=6, P>0.05) nor the steady state (I/Iₒ=1.01±0.03, n=6, P>0.05) NMDA responses(FIG. 1B).

GBP potentiated NMDA responses in cells with elevated PKC. The effects of GBP on NMDA responses were then tested in cells with elevated PKC. PKCM (a catalytically active form of PKC) was included in the patch pipette solution and measured the NMDA response before and after the addition of GBP. As PKCM diffused into the cell following the rupture of the membrane patch in the whole-cell recording mode, peak NMDA responses gradually increased. In addition, the NMDA-activated currents desensitized at faster rates. This was evident by the minimal increase in the steady state currents. The enhancement of peak NMDA responses was 150.0±9.0% and of the steady state NMDA responses was 95.0±9.0 (n=5). These observations were similar to our earlier study on trigeminal neurons (Chen et al., Nature 356:521-523 (1992)) and others on hippocampal neurons (Xiong et al., Mol. Pharmacol., 54:1055-63 (1998)).

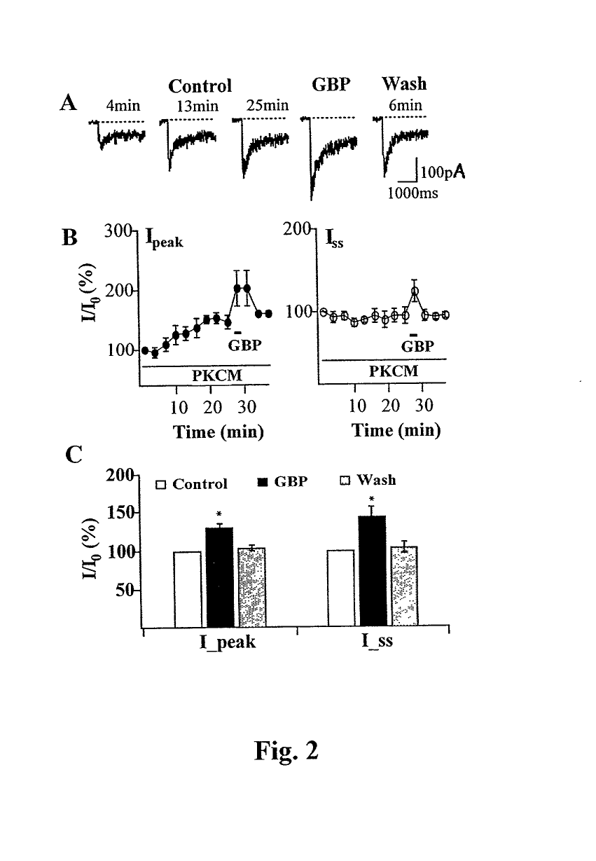

After the NMDA responses had reached a steady state level, GBP was applied to the cell (FIG. 2). In the presence of PKCM, GBP increased both the peak and steady state NMDA currents. The NMDA currents recovered to the control level when GBP was removed from the external solution. In addition, GBP further increased the decay of the NMDA current. The fast decay time constants (τ) were 0.26±0.03 ms (n=9) in PKC and 0.12±0.009 (n=9) in GBP (P<0.01, student test). The changes in NMDA amplitudes are summarized in FIG. 2C. GBP increased peak NMDA currents by 34% (I/Iₒ=1.34±0.06, n=9) and steady state currents by 46% (I/Iₒ=1.46±0.12, n=9).

To determine the specificity of PKCM, two control experiments were performed. First, the PKCM solution was boiled for 3-5 minutes to denature the peptide and repeated the same experiment as in FIG. 2. In the presence of denatured PKCM, GBP had no effect on NMDA current responses (n=6). In the second control experiment, only the vehicle used to stabilize PKCM in the patch pipette was included. GBP could not enhance NMDA-activated currents in the presence of the vehicle (n=10). Thus, PKC indeed exerted specific effects on dorsal horn cells.

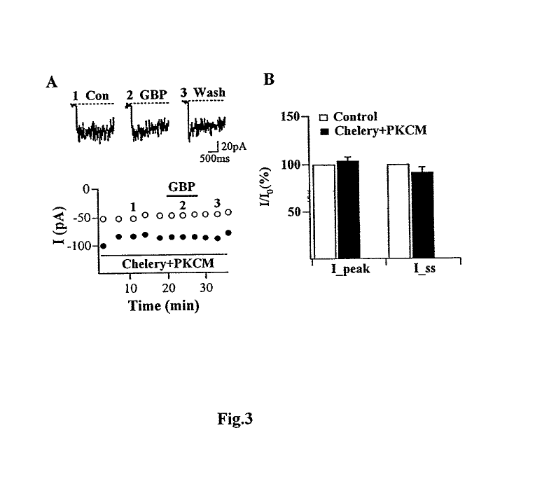

To further confirm that GBP potentiation of NMDA responses is specifically related to the levels of PKC, the PKC inhibitor, chelerythrine, was added to the external solution and the experiments were repeated as described in FIG. 2. In the presence of 6.6 μM chelerythrine, PKCM did not increase the NMDA responses, suggesting that chelerythrine (6.6 μM) completely blocked the action of PKCM on NMDA-activated currents (FIG. 3). Under these conditions, GBP no longer potentiated NMDA responses (peak I/Iₒ=1.04±0.04, steady state I/Iₒ=0.92±0.06, n=10, P>0.05). Thus, it is concluded that GBP enhances NMDA currents when the level of PKC inside the cells was elevated.

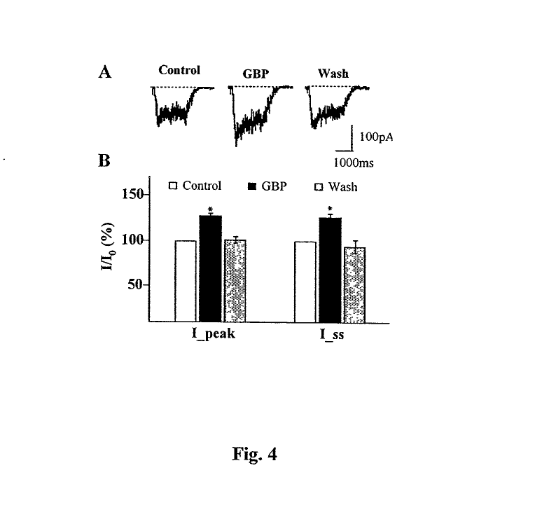

GBP increased NMDA responses in neurons isolated from inflamed rats without PKC treatment. If the hypothesis that NMDA responses to GBP depend on the level of PKC was correct, GBP should enhance NMDA responses in neurons isolated from inflamed rats without any PKC treatment because the levels of PKC, particularly PKC, in these neurons are elevated (Martin et al., Neuroscience, 88:1267-74 (1999)). To test this, the GBP actions on NMDA-activated currents in neurons isolated from CFA-treated rats were examined. Injection of CFA into the hindpaw of the rats caused erythema, edema and hyperalgesia (Guo et al., Soc. Neurosci. Abstr., 25:920 (1999). These inflammatory responses became prominent one day after CFA treatment and the hyperalgesic conditions lasted for 2-3 weeks (Martin et al., Neuroscience, 88:1267-74 (1999)). The effects of GBP on NMDA-activated currents were studied in cells isolated from rats 2-7 days after the injection of CFA. In contrast to the untreated cells in which GBP had minimal effects on NMDA responses (FIG. 1), GBP potentiated NMDA-activated in 56% (19 out of a total of 34) cells examined. The magnitude of enhancement for peak currents was 28.0±2.7% and for steady state currents was 27.0±3.3% (n=19) (P<0.05) (FIG. 4). The significant increase in the cells responding to GBP is consistent with the idea that GBP acts only on NMDA receptors in cells with elevated PKC.

GBP increased NMDA responses by altering the glycine affinity for NMDA receptors. To determine the mechanism of action of GBP, the effects of GBP in different concentrations of glycine in the presence of PKCM was examined. Glycine is a co-activator of NMDA receptor that binds to the receptor with high affinities. To make sure that adding GBP did not change the basal glycine concentrations of our external solutions, the glycine content of GBP external solutions was measured. The basal glycine of GBP-containing solution was 30.0±2.0 nM (n=3), a level similar to that found in control solutions.

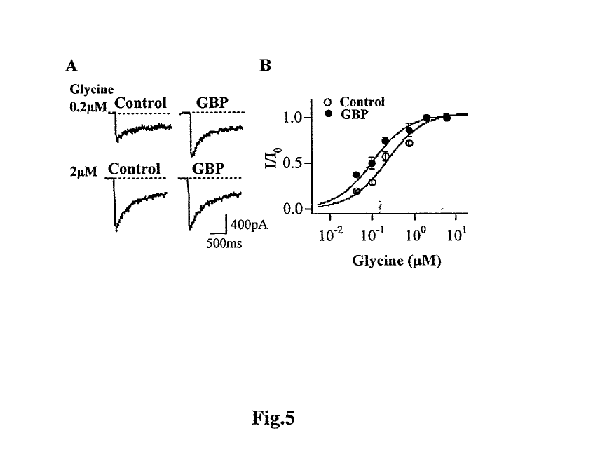

With saturated glycine (2 μM), GBP did not potentiate NMDA response (FIG. 5A). With 0.2 μM external glycine, GBP enhanced peak NMDA currents by 43% in the same cell (FIG. 5A). When the external glycine concentration was lowered to 0.1 μM, the potentiating effect of GBP increased up to 80.0±17.0% (n=3) (FIG. 5B).

To determine whether GBP altered the affinity of glycine for NMDA receptors, the effect of GBP on NMDA responses were studied at a series concentrations of glycine (FIG. 5B). The dose-response curves for glycine with and without GBP were plotted. Under the control conditions, the EC₅₀ of glycine for NMDA receptors was 0.2 μM. In the presence of 50 μM GBP, the EC₅₀ was reduced to 0.09 μM (FIG. 5B). Thus, GBP increased the glycine affinity for NMDA receptors.

In conclusion, this evidence shows that GBP had no effect on NMDA-evoked currents under normal conditions but potentiated NMDA responses when PKC inside cells is elevated. This action of GBP was PKC-specific because it is blocked by the PKC inhibitor, chelerythrine (FIG. 3). This plastic action of GBP is further supported by the study of GBP in neurons isolated from inflamed rats (FIG. 4). In contrast to normal cells where GBP potentiates NMDA responses only in the presence of exogenous PKC, GBP exerts its effects on inflamed neurons without any PKC treatment (FIG. 4).

Following tissue or nerve injury, dorsal horn neurons develop hypersensitivity to innocuous (allodynia) and noxious (hyperalgesia) stimuli. These pathologic conditions are closely linked to the activation of NMDA receptors and the elevation of PKC. It is well established that sensitization of dorsal horn neurons following tissue injuries cannot be initiated or maintained when the activation of NMDA receptors is blocked by NMDA receptor antagonists (Woolf et al., Pain 44:293-9 (1991)). Anatomical studies show that immunoreactivity for PKC, particularly the PKCγ isoform, in dorsal horn neurons increases by 75-100% in rats with inflammation or nerve injuries (Martin et al., Neuroscience, 88:1267-74 (1999)). Although responding to acute nociceptive stimuli normally, mutant mice with a deletion of the PKCγ gene develop only mild allodynia with partial sciatic nerve injury (Basbaum, Reg. Anesth. Pain Med., 24:59-67 (1999)). The properties of NMDA receptors are profoundly affected by increasing levels of PKC elicited by injuries. For example, capsaicin-induced inflammation elicits increases in PKC and the levels of phosphorylated NMDA receptors in spinothalamatic neurons (Zou et al., Soc. Neurosci. Abstr., 25:1980 (1999). The current-voltage curve of NMDA-evoked currents in CFA-induced inflamed rats shifts to the hyperpolarized direction and the shift is blocked by protein kinase inhibitors (Guo et al., Soc. Neurosci. Abstr., 25:920 (1999). In view of the evidence presented herein, these observations support the conclusion that GBP potentiates NMDA responses in inflamed rats as a result of high levels of endogenous PKC.

Subcutaneous injection of formalin into the paw elicits two phases of licking, biting and flinching behaviors in rats. The first transient phase of nociceptive behaviors lasts <10 minutes and is followed by a second sustained phase of nociceptive behaviors lasting for ˜50 minutes. It has been found that GBP has no effect on the nociceptive behaviors in the transient phase while potently blocking the sustained phase of the formalin-induced nociceptive responses (Carlton et al., Pain 76:201-7 (1998); and Shimoyama et al., Neurosci. Lett., 222:65-7 (1997)). It is concluded that GBP affects sustained nociceptive responses in the formalin test because NMDA receptors mediate the sustained responses and the endogenous PKC levels during this phase are high. There is strong evidence supporting this suggestion. It has been shown that formalin treatment triggers intense activation of C-fiber afferent fibers (Heapy et al., Br. J. Phannacol., 90:164P (1987)). The NMDA receptor antagonist inhibits neuronal activity only during the sustained phase of the formalin test (Coderre et al., J. Neurosci. 12:3665-70 (1992); Coderre, Neurosci. Lett., 140:181-4 (1992) and Haley et al., Brain Res., 518:218-26 (1990)). In addition, treatment of PKC inhibitor reverses the sustained, but not the acute, phase of the formalin responses.

A large percentage of cells studied were from superficial laminae and more than 80% of them responded to GBP in the presence of PKC. Thus, this evidence suggests that GBP can potentiate NMDA responses of superficial neurons as well as those of deep neurons when the PKC level is elevated.

The site of GBP action also is determined here. Because the dose-response curve for glycine shifts to left with GBP (FIG. 5), this suggests that GBP interacts with the glycine site on NMDA receptor. Since NMDA responses saturate at high concentrations of glycine, no further increase by GBP should be observed under these conditions. This explains the behavioral observations that application of the glycine receptor agonist, D-serine, blocks the antinociceptive effects of GBP (Singh et al., Psychopharmacology (Berl) 127:1-9 (1996); Yoon et al., Anesth. Analg., 89:434-9 (1999)) and also explains the observation that GBP could not enhance NMDA responses in 1 μM glycine (Rock et al., Epilepsy Res., 16:89-98 (1993)).

One likely mechanism for how the enhancement of NMDA responses by GBP results in antinociception could be that GBP affects mostly inhibitory intemeurons. An increase in NMDA responses by GBP would promote the activity of inhibitory neurons and thus result in a reduction of transmission of nociceptive signals. Preliminary studies of the GABA content of the recorded cells isolated from inflamed rats support this idea. Most cells (80%) responding to GBP were found to be GABA-positive, while those that do not respond to GBP are not immunoreactive for GABA.

This study of GBP actions on NMDA receptors leads to the conclusion that GBP action does not remain constant but depends on the state of postsynaptic cells and receptors. At low levels of PKC, GBP would not affect NMDA receptors. Only when PKC is elevated does GBP exert its effects. This property of a drug is particularly desirable because it affects only those cells that are injured leaving those healthy ones alone. This would limit drug actions to specific areas of the central nervous system affected by inflammation or nerve injuries and keep side effects to a minimum. These results also point to a new strategy for drug design. A chemical whose effect depends on the state of a receptor molecule will not only give more specific spatial actions, but also will be temporally selective for specific cell conditions.

The complete disclosure of all patents, patent applications, and publications, and electronically available material (e.g., GenBank amino acid and nucleotide sequence submissions) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims. All headings are for the convenience of the reader and should not be used to limit the meaning of the text that follows the heading, unless so specified.

What is claimed is:

1. A method for identifying an agent that increases an N-Methyl-D-Aspartate (NMDA) receptor mediated response in a neuron to an agonist comprising contacting the neuron with the agent, the neuron comprising an NMDA receptor and an elevated level of protein kinase C (PKC), and determining whether the NMDA receptor mediated response in the neuron contacted with the agent is altered relative to a neuron not contacted with the agent.

2. The method of claim 1 wherein the neuron is ex vivo.

3. The method of claim 2 wherein the neuron is isolated from a spinal cord.

4. The method of claim 3 wherein the neuron is isolated from a dorsal horn.

5. The method of claim 4 wherein the neuron is isolated from the area of the dorsal horn selected from the group consisting of lamina I, lamina II, lamina IV, and lamina V.

6. The method of claim 3 wherein the neuron is isolated from a central canal.

7. The method of claim 6 wherein the neuron is isolated from lamina X of the central canal.

8. The method of claim 2 wherein the neuron is isolated from a brain.

9. The method of claim 8 wherein the neuron is isolated from a trigeminal subnuclear caudalis.

10. The method of claim 1 wherein the neuron is in vitro.

11. The method of claim 10 wherein the neuron is a human neuroblastoma neuron.

12. The method of claim 11 wherein the neuron is selected from the group consisting of NG108-15, N1E-115, and SHSY5Y.

13. The method of claim 1 wherein the agonist is selected from the group consisting of NMDA, glutamate, and aspartate.

14. The method of claim 1 further comprising determining whether the agent increases the affinity of glycine for the NMDA receptor.

15. A method for identifying an agent that, in an animal, reduces pain from a neuropathological condition, the method comprising contacting a neuron with the agent, the neuron comprising an NMDA receptor and an elevated level of PKC, and determining whether the NMDA receptor mediated response in the neuron contacted with the agent is altered relative to a neuron not contacted with the agent.

16. The method of claim 15 wherein the neuropathological condition is selected from the group consisting of peripheral nerve injury, postherpetic neuralgia, diabetic neuropathy, trigeminal neuralgia, and cancer.

17. The method of claim 15 wherein the neuron is ex vivo.

18. The method of claim 15 wherein the neuron is in vivo.

19. The method of claim 18 wherein the alteration in the NMDA receptor mediated response is measured by evaluating a change in allodynia in an animal.

20. The method of claim 18 wherein the alteration in the NMDA receptor mediated response is measured by evaluating a change in hyperalgesia in an animal
